(19) United States
(12) Patent Application Publication
(10) Pub. No.: US 2016/0299473 A1
(43) Pub. Date: Oct. 13, 2016

(54) Cosmetics Spoilage and Past Due Detection Monitoring System Organizer

(71) Applicant: Karla Solis Zuniga, Plano, TX (US)

(72) Inventor: Karla Solis Zuniga, Plano, TX (US)

(21) Appl. No.: 14/684,376

(22) Filed: Apr. 11, 2015

(51) Int. Cl. G04F 1/00; G04G 21/02; A61L 2/10

(52) U.S. Cl. CPC G04F 1/005; A61L 2/10; G04G 21/02

(57) ABSTRACT

This invention relates to a cosmetics spoilage and past due detection monitoring system organizer that has integrated user activated timing devices and is used as a recommended cosmetic use monitor for providing an indication of when the recommended usable time frame of cosmetic products has expired. The invention consists of an organizational device having a plurality of cosmetics storage compartments. Each storage compartment houses designated cosmetic products and has an associated corresponding timing device that monitors the designated individual cosmetic product's recommended usable time frame. What is unique is that the physical presence of the cosmetic product is directly and visibly tied to its recommended past due or expiration date within the cosmetic products' assigned compartment and therefore is advantageous in reducing diseases.

### CROSS-REFERENCE TO RELATED APPLICATION

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### FIELD OF THE INVENTION

This invention pertains or relates to the field of cosmetics and safe cosmetics storage specifically the invention pertains to a cosmetic spoilage and past due detection and monitoring system.

### BACKGROUND OF THE INVENTION

A major problem facing users of cosmetic products are the health dangers of using cosmetic products beyond the manufacture's recommended usage time frames. It is well know that many cosmetic products such as mascara that have been used beyond the manufacture's recommended usage date have been found to have staphylococcus, streptococcus bacteria, harmful fungi, and infection causing bacteria such as those bacteria leading to “pink eye.” The International Journal of Cosmetic Science has reported that serious adverse reactions can occur following the use of expired makeup including tearing, erythema, itchiness, irritation, and allergy. The further a cosmetic product is used past the cosmetic manufacture's recommended use date, the higher the likelihood of increased levels of aerobic bacteria thus causing an increased chance of the user contracting skin diseases or eye diseases that could potentially lead to blindness.

It is crucially important to track the time that has elapsed since the initial purchase and/or use of a particular cosmetic product against the product manufactures' or scientific community's recommended expiration or past due date. Similarly, it is critically important that once the past due date of a cosmetic product has been exceeded that the cosmetic user be notified that the product is potentially unsafe for use. This invention integrates the tracking, monitoring, and alerting functionality within the new apparatus by providing user settable timers which countdown the number of usage days left for each compartment's cosmetic product and then once the past due date has been exceeded, the apparatus then notifies the user audibly and/or visually that the cosmetic product has exceeded its expiration date.

Since the Food and Drug Administration does not require expiration dates on cosmetic products, many cosmetic products users take the extra step of determining the cosmetic product's expiration dates by researching manufacture information based on products' batch number and other available cosmetic product information to the cosmetic user. Moreover, these same cosmetic users take into consideration the recommended usage time frames of cosmetics products from experts outside of the cosmetic industry such as those in the medical and scientific community. Even with the correct determination of the cosmetic product expiration date the cosmetic user does not currently have a user interactive system that will associate various cosmetic products' physical presence with that of that individual cosmetic product's expiration date.

This invention can keep track of, monitor, and alert the user once the particular cosmetic products' recommended usage time frame has been exceeded satisfying that important need which is to associate the various cosmetic product's physical presence with that of that cosmetic product's past due date. Without the invention this information on the time period of safe use for the various cosmetic products will likely be forgotten. The embodiments of the invention allow the cosmetic user to be able to select and set a countdown timer of safe usage for each cosmetic product or a group of similar cosmetic products within its own compartment within the system. When the user reaches for a particular cosmetic product, the user can easily identify the remaining number of the days of safe usage left for the product assisting the cosmetic user in reducing the amount of bacteria they are exposed to in an effort to prevent eye and skin infections and even potential blindness.

Another advantage of the invention is that once the user enters the recommended past due date for that particular product within the system, this negates the need for special individual product packaging with the same information and further allows the user to keep track of the past due date even if the labeling communicating the individual cosmetic product's past due date or expiration date wears off as frequently happens today.

Another monitoring feature of the embodiments of the invention is that the system integrates designs and embodiments that have thermometers and internal heat sensors to warn the cosmetic user when spoilage of cosmetic products may have occurred because the cosmetic manufacture's recommended safe storage temperature has been exceeded.

Besides just monitoring and alerting the cosmetic user of a products past due date being exceeded, the embodiments of the invention do not stop there, the invention also mitigates the grown of harmful bacteria and breakdown of chemical compounds within the cosmetic products by providing optional ultraviolet (UV) tint on the enclosed embodiments of the invention that are made out of translucent material. Embodiments of the invention also integrate commercially available heat sensors that can record the highest temperature reached. The enclosed embodiments of the invention will employ these highest temperature reached heat sensors to alert the user of the highest temperature reached and will identify which cosmetic product's highest safe storage temperature has been exceeded. Some of the compartments can be organized by color coding denoting the highest safe temperatures allowed for those cosmetic products within the compartment.

Further, desiccant is integrated within the enclosed embodiments of the invention to reduce water and moisture which is a major constituent of living material such as harmful bacteria which participates in many metabolic reactions several of which reduce the safety usage of the cosmetics. Water and extra humidity also catalyze the chemical decomposition of many chemical compounds comprising the cosmetic products.

For the foregoing reasons, there is a need for a cosmetics spoilage and past due detection and monitoring system organizer that not only allows for the organization and storage of cosmetic products but additionally through a plurality of user activate integrated timing devices identifies when individual cosmetic products are spoiled or beyond the cosmetic manufactures' recommended use date for the particular cosmetic product and thereby reduces the risk of skin, eye, and infectious diseases.

### DESCRIPTION OF PRIOR ART

There are a plurality of cosmetic organizers but none can be found which has integrated timing devices which can be individually set by the cosmetic user to keep track of when the recommended usable time frame of the particular cosmetic product has ended and ties the physical presence of the particular cosmetic product to its associated expiration date for all cosmetic products within the invention.

### SUMMARY

The present invention is directed to a cosmetics spoilage and past due detection monitoring system organizer that tracks the time that has elapsed since the initial purchase and/or use of a particular cosmetic product against the product manufactures' or scientific community's recommended expiration or past due date and then once the expiration date of a cosmetic product has been expired, notifies the cosmetic user audibly and/or visually that the cosmetic product is potentially unsafe for use. The enclosed to outside environment embodiments of the invention will have desiccant integrated within the system to inhibit or restrict the growth of bacteria by lowering the humidity and for the enclosed versions made out of translucent material will have ultra-violet tint to reduce the temperature of the system organizer and therefore reduce the chemical breakdown of the cosmetic products.

Thus, the invention will reduce the risk of eye infections, skin infections, potential blindness, and other diseases associated with the use of bacteria ridden and chemically degraded cosmetics products. Chemical decomposition is a natural process with occurs over a period of time when subjected to normal environmental conditions such as heat, radiation, humidity and the acidity of the compound itself. Often the chemical decomposition is an undesirable chemical reaction. Almost all cosmetics are made up of chemical compounds which are continually decomposing and the cosmetic user needs to pay special attention to its expiration date to avoid harmful effects and physical injury.

There are many factors of the invention that make it new and advantageous. First is the fact that the physical presence of the cosmetic product is directly and visibly tied to its recommended past due or expiration date. This is accomplished because within the system there is a plurality of compartments housing the individual cosmetic products and each individual compartment has associated corresponding timing devices that monitor the designated cosmetic product's recommended usable time period. The individual cosmetic product's expiration date will be communicated by the system to the user by a visual and/or audio indication when the product's past due date has expired.

An example would be a user having two very similar shades of red lipstick each with a different expiration date. The user would have one shade of the red lipstick in its own separate compartment with that same compartment having its own separate timer showing the number of days left to expiration that may have been worn off the product. Likewise the other similar shade of red lipstick would have its own separate compartment with its own separate timer showing the number of days left to expiration. Thus, the invention if used properly would preclude the cosmetic user from getting the two lipstick's past due dates mixed up. The same tie of physical presence association of the cosmetic product to its expiration date applies to all cosmetic products within the invention. This tie of physical presence of the cosmetic products within the plurality of the individual compartments each to its associated expiration date with the organizer that the cosmetic user is able to individually set and monitor is unique and advantageous in reducing diseases.

As already discussed, the diseases associated with spoiled and past due expiration dates of mascara can be very serious and can even lead to blindness. Many mascara products look very similar and just thrown loose inside a purse, on the floor of the car, the glove box of the car, open drawer, upon the counter, or on the floor of a room, the mascara's expirations date will get mixed up or even forgotten. Once the mascara is entered in the invention's appropriate compartment with its own separate expiration date, the user could see at a glance whether the mascara is recommended safe, past due, or significantly past due and that the user would be reminded of the fact before applying the potentially bacteria-ridden or chemically decomposed mascara upon the user's eyes. It could very well be a sight saving last second reminder for the user.

The second new and advantageous factor of the invention is that it provides an additional means of storage of the cosmetics products' expiration date. Many times the user of the cosmetic product may temporarily have the cosmetic product away from the invention, such as in the user's purse or out on the beach and if there is an expiration date on the cosmetic product itself, this date can rub or peel off during use of the cosmetic product. Upon returning the cosmetic product to the invention, the expiration date will once again be associated with the physical presence of the cosmetic product. This further allows all the cosmetic user's cosmetic products to be monitored without the need for special individual cosmetic product packaging or when the products expiration date has been worn off.

Third, the invention allows for optional desiccant to be integrated within the enclosed embodiments types on the invention to assist in inhibiting the presence of extra moisture which would otherwise proliferate the growth of harmful bacteria and the chemical decomposition of the chemical compounds making up the cosmetic products.

Fourth, the invention allows for optional UV tint to be integrated within the enclosed embodiment types on the invention made out of translucent material to reduce the internal temperature of the system assisting in inhibiting the growth of harmful bacteria and in reducing the rate of decomposition of the chemical compounds comprising the cosmetic product.

Fifth, the invention allows for optional thermometers and/or thermal sensors to be integrated within the enclosed embodiment types on the invention to increase the user's monitoring ability of the system which would alert the user if a recommended safe temperature for the product was exceeded.

Sixth, the invention allows for optional heat sensors to be integrated within the enclosed embodiment types on the invention to increase the user's monitoring ability of the system and if a recommended safe temperature for the product was exceeded to audibly and/or visually alert the user upon the users return to the system.

Seventh, the invention allows for optional UV germicidal irradiation lamps within the enclosed embodiment types on the invention to sterilize the internal surfaces of the compartments.

For the foregoing reasons, there is a need for a cosmetics spoilage and past due detection and monitoring system organizer that ties of physical presence of the cosmetic products within the plurality of the individual compartments each to its associated expiration date within the organizer that the cosmetic user is able to individually set and monitor when individual cosmetic products are spoiled or beyond the cosmetic manufactures' recommended use date. Thus, this invention reduces the risk of skin and eye infection, potential blindness, and other infectious diseases.

### ASPECTS OF THE INVENTION

Accordingly, it is an aspect of the invention to provide a cosmetic system organizer with integrated timing devices which when set to count down the remaining safe usage days of manufactures' recommended usage time frames, will reduce the user's chance of contracting skin, eye and other diseases when the product is not used past its recommended safety date.

Still further another aspect of the invention is to effect the previous objects with a cosmetic organizer that takes on any of the following embodiments: cosmetic organizer without cover, enclosed cosmetic organizer with cover, enclosed insulated cosmetic organizer with cover, cosmetic organizer containing a variety of cosmetic storage devices to accommodate the storage needs of users, enclosed insulated cosmetic organizer with cover and place to put cooling bag or coolant bars, cosmetic organizer with drawers, cosmetic organizer bag.

Still another aspect of the invention is to effect the previous objects to assist with reducing the risk of cosmetic spoilage within the system organizer by having commercially available desiccant integrated within the enclosed version of invention so as to inhibit or restrict the growth of bacteria by lowering the ambient humidity.

Still another aspect of the invention is to prevent breakdown and spoilage of cosmetic products to have ultraviolet tint on the variations of the invention that are made of visibly translucent materials to reduce the temperature of the system organizer and chemical breakdown of the cosmetic products.

Still another aspect of the invention is to have UV germicidal irradiation lamps that irradiate and kill harmful bacteria.

Still another aspect of the invention is to have thermometers and internal heat sensors to warn the cosmetic user when spoilage of cosmetic products may have occurred.

These and other features, aspects, and advantages of the present invention will become better understood with regard to the following description, appended claims and accompany drawings.

### BRIEF DESCRIPTION OF THE DRAWING FIGURES

FIG. 1 is a perspective view of one embodiment according to the cosmetic spoilage detection and past due monitoring system organizer of the present invention

FIG. 2 is a modular embodiment of the invention.

FIG. 3A is a perspective view of a desiccant sheet and desiccant holder in a detached state.

FIG. 3B is a perspective view of a desiccant sheet attached to the underside of the desiccant holder.

FIG. 3C is a perspective view of depicting one exemplary installation of the desiccant holder into one embodiment of the invention.

FIG. 4 is a perspective view of an open embodiment of the present invention.

FIG. 5 is a perspective view of an insulated modular embodiment of the present invention.

FIG. 6 is a perspective view of one way multiple modular drawers can be arranged.

### REFERENCE LISTING OF THE NUMBERED ELEMENTS

110 Plurality of Multiple Storage Holding Compartments
111 Drawer
112 Drawer Support Base
113 Drawer First Wall
114 Drawer Second Wall
115 Drawer Third Wall
116 Drawer Fourth Wall
117 Drawer Internal Storage Compartment
118 Drawer Handles
120 Electronically Controlled Timers
121 Electronically Controlled Timer Display
122 Electronically Controlled Timer Visual/Audio Indicator
123 Electronically Controlled Timer Start/Reset Button
130 Thermometer/Thermal Sensor
131 Thermometer/Thermal Sensor Display
132 Thermometer/Thermal Sensor Visual/Audio Indicator
133 Thermometer/Thermal Sensor Start/Reset Button
160 Desiccant/Desiccant Sheet/Desiccant Packs
161 Adhesive Area
170 Desiccant Holder
171 Perforations
180 Air Flow Gap
190 Mirror
191 Mirror Holder
210 Base
211 Frame
212 Rigid Dividers
220 Circumferential Ledge
230 Visual/audio Indicator
240 Ultraviolet Tint
250 Embodiment of Cosmetic Organizer Closed System
260 Electronic Device Holder
261 Electronic Device
262 Electronic Device Holder Aperture
270 Floor of the Plurality of Cosmetic Compartments
280 Embodiment of Modular Drawer
281 Modular Drawer Support Base
282 Modular Drawer First Wall
283 Modular Drawer Second Wall
284 Modular Drawer Third Wall
285 Modular Drawer Fourth Wall
286 Modular Drawer Internal Storage Compartment
290 Ultraviolet Germicidal Irradiation Lamps
291 Ultraviolet Germicidal Irradiation Lamp Power Source
292 Ultraviolet Germicidal Irradiation Lamp On/Off Switch
300 Open Embodiment of Cosmetic Organizer System
301 Timer Mounting Area
400 Embodiment of Insulated Modular Drawer
401 Insulated Drawer Encasement External First Wall
402 Insulated Drawer Encasement External Second Wall
403 Insulated Drawer Encasement External Third Wall
404 Insulated Drawer Encasement Overhead Wall
405 Insulated Drawer Encasement Support Base
406 Insulated Drawer Encasement Internal First Wall
407 Insulated Drawer Encasement Internal Second Wall
408 Insulated Drawer Encasement Internal Third Wall
409 Enclosed Insulation Cavity
410 Insulation Bead
411 Drawer Compartment
412 Visual Identifications

### DEFINITIONS

The System—Cosmetic Spoilage Detection and Past Due Monitoring System Organizer,

Desiccant—desiccant that can be loose desiccant, desiccant packs, desiccant sheets.

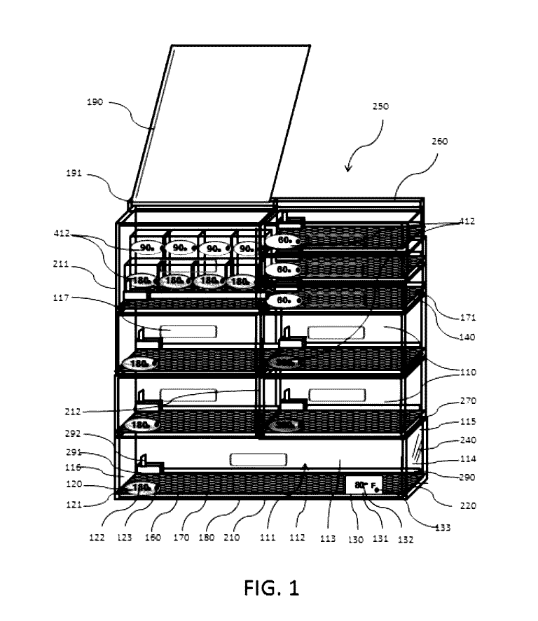

Referring generally to FIG. 1, an enclosed embodiment cosmetics organizer 250 comprised of a base 210, frame 211, rigid dividers 212, and drawers 111. These parts can be fabricated using a variety of assembly procedures known to those skilled in the art and can be constructed of rigid material substances such as acrylic, metal, wood, or translucent materials. The frame 211 and dividers 212 can form combinations of similarly-sized and differently-sizes storage compartments 110. Frame 211 can be constructed to allow for a plurality of multiple storage compartments 110 as shown in FIG. 1. Cosmetic organizer 250 is further provided with rigid storage drawers 111. Drawer 111 comprises of a support base 112, a first wall 113, a second wall 114, a third wall 115, and a fourth wall 116. The first wall 113, second wall 114, third wall 115, and fourth wall 116 extend upward from the support base 112 defining storage compartment 110. Drawer 111 has a drawer handle 117 used to pull the drawer 111 frame 211 from the frame.

Drawer 111 has an internal circumferential ledge 220 close to the support base 112 that allows a desiccant holder 170 to rest slightly above the support base 112 to allow an air flow space 180 between the support base 112 and the desiccant holder 170. The desiccant holder 170 can hold desiccant 140 in the form of desiccant sheets 160 and has perforations 171 to allow airflow for improved moisture removal. In an embodiment of the drawer 111, an electronically controlled timer 120 is mounted to the first wall 113. The electronically controlled timer 120 can indicate the number of days, weeks, months, or years that have passed. The electronically controlled timer 120 can count up or down a specific preset number of days, weeks, months, or years that are left in regards to the recommended usage timeframe of cosmetic products.

The electronically controlled timer 120 has a user-activated start/reset button 123. The electronically controlled timer 120 is programmed so that upon receipt of current from a power source such as a battery, a signal is provided to the display 121. The display 121 includes a visual/audio digital indicator 122. The visual/audio digital indicator 122 is electronically operated, and is preferably part of a liquid crystal display (LCD), but could alternatively be provided as other types of displays such as light emitting diodes (LEDs). The digital indicator displays preferably one, two, and three digits which correspond to the number of days, weeks, months, and years remaining in the useful life of the cosmetics products being monitored by the electronically controlled timer 120.

Furthermore, to facilitate identification of the recommended usage time frame in addition to the pre-set time displayed on the electronically controlled timer 120, each electronically controlled timer 120 can be color coded to provide visual identifications 412 for each time frame and/or cosmetic product category being monitored. A color coding chart identifying the different recommended use timeframes may be provided with each embodiment of the present invention to assist in identification of which multiple storage compartment a particular cosmetic product needs to be stored.

FIG. 1 further comprises a thermometer 130 which includes a display 131, a visual/audio indicator 132, and start/reset button 133. The thermometer 130 measures ambient temperature and has a means to trigger a visual and/or audio indicator 132 when the recommended storage temperature has been exceeded. The thermometer 130 can be attached to the first wall 113 of any drawer 111. Drawer 111 further includes an electronically controlled ultraviolet germicidal irradiation lamp 290. The electronically controlled ultraviolet germicidal irradiation lamp 290 can have a battery power source 291 that has an On/Off switch 292. The frame 211 and drawers 111 can have Ultraviolet (UV) tint 250 to offer UV protection to the cosmetic products stored within the container. Other UV tinting procedures and techniques known to those skilled in the art can also be applied.

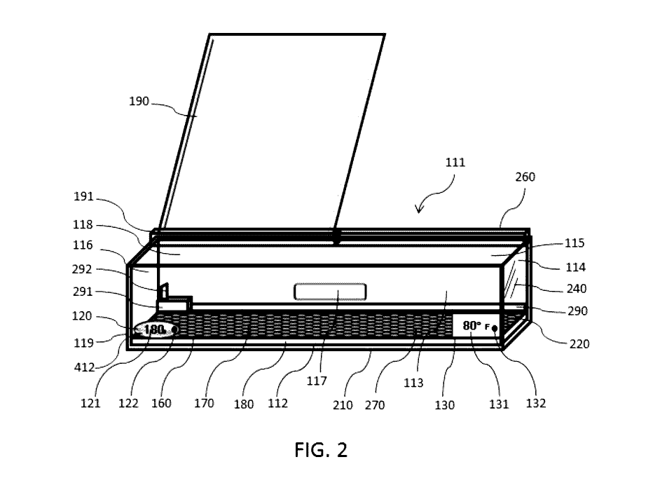

FIG. 1 shows an embodiment further comprising a mirror 190 and mirror holder 191. FIG. 1 further comprises an electronic device holder 260. The electronic device holder 260 and mirror holder 191 are attached to the top of the frame 211. FIG. 2 is a perspective view of the modular drawer embodiment 280 of the present invention. As shown in FIG. 2, the modular drawer 280 comprises a support base 281, a first wall 282, a second wall 283, a third wall 284, and a fourth wall 285. The first wall 282, second wall 283, third wall 284, and fourth wall 285 extend upward from a first side of the support base 281. Modular drawer 280 has a drawer handle 287 on wall first wall 282 used to pull out the modular drawer 280 from the external modular drawer encasement 288. The modular drawer 280 contains a desiccant holder 170 that sits on a circumferential ledge 220 above the support base 281 creating an airflow space 180 between the desiccant holder 170 and the support base 281. The desiccant holder 170 has perforations 171 that allow the desiccant 140 to absorb moisture.

FIG. 2 shows an electronically controlled timer 120 and a thermometer/thermal sensor 130 affixed to the first wall 282. The modular drawer 280 contains an electronically controlled ultraviolet germicidal irradiation lamp 290 within the internal storage compartment 286. FIG. 2 further shows an embodiment comprising a mirror 190, mirror holder 191, and an electronic device holder 260. The electronic device holder 260 and mirror holder 191 are attached to the top of the external modular drawer encasement 288.

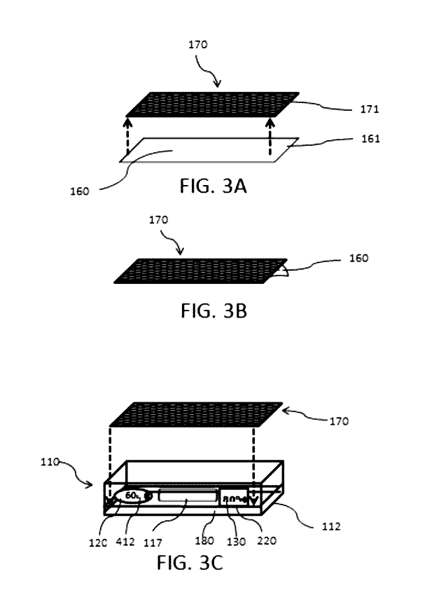

FIG. 3A is a perspective view of the desiccant holder 170 and desiccant sheet 160 in a detached state. Desiccant sheet 160 may have an adhesive area 161 that allows it to attach to the underside of the desiccant holder 170. Desiccant holder 170 has perforations 171 throughout to allow the desiccant sheet 160 to absorb moisture. The desiccant holder 170 can be constructed from rigid material substances such as acrylic, metal, wood, and glass.

FIG. 3B is a perspective view of the desiccant holder 170 with a desiccant sheet 160 attached to the underside of the desiccant holder 170.

FIG. 3C is a perspective view of one way to assemble the desiccant holder 170 into the multiple storage holding compartments 110. The desiccant holder 170 can be placed inside the multiple storage compartments 110 and can rest on the circumferential ledge 220. An air flow gap 180 exists between the support base 112 and the desiccant holder 170 to allow the desiccant sheet 160 to absorb moisture.

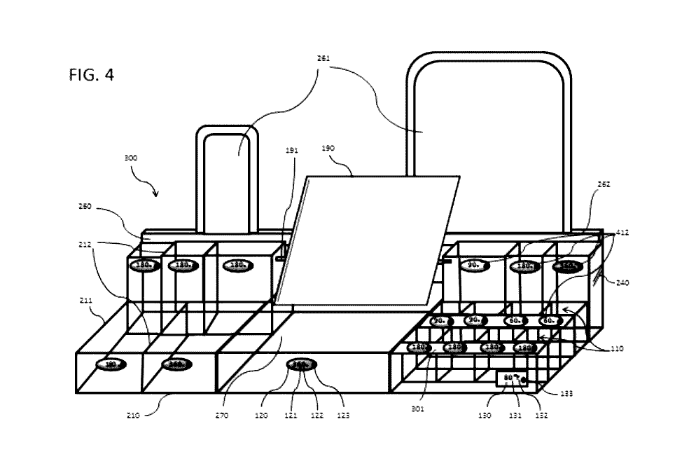

FIG. 4 is a perspective view of one embodiment according to the cosmetic spoilage detection and past due monitoring system organizer of the present invention.

FIG. 4 shows another embodiment of the present invention, the open embodiment cosmetics organizer 300 comprised of a base 210, frame 211, and rigid dividers 212. These parts can be fabricated using a variety of assembly procedures known to those skilled in the art.

The frame 211 and dividers 212 can form combinations of similarly-sized and differently-sizes storage compartments 110. Frame 211 can be constructed to allow for a plurality of multiple storage compartments 110 as shown in FIG. 4.

In this open embodiment cosmetic organizer 300, electronically controlled timers 120 comprising of a display 121, visual/audio indicator 122, and start/reset button 123, are mounted to the front-facing side of the frame 211 and also on the timer mounting area 301. This arrangement of timers 120 allows users to identify the corresponding timer 120 to each of the multiple storage holding compartments 110. FIG. 4 shows a thermometer 130 comprising of a display 131, visual/audio indicator 132, and start/reset button 133, attached to the front-facing side of the frame 211. This embodiment comprises of UV tint 240 on the frame 211 and rigid dividers 212.

FIG. 4 shows an embodiment further comprising an electronic device holder 260 shown holding two electronic devices 261. The electronic device holder 260 has an aperture 262 to allow it to hold electronic devices 261. The electronic device holder 260 is attached to the back side of the frame 211. FIG. 4 further comprises a mirror 190 held in position by a mirror holder 191 that is attached to the frame 211. The mirror 190 may have a swivel mechanism to allow users to adjust the angle of the mirror 190.

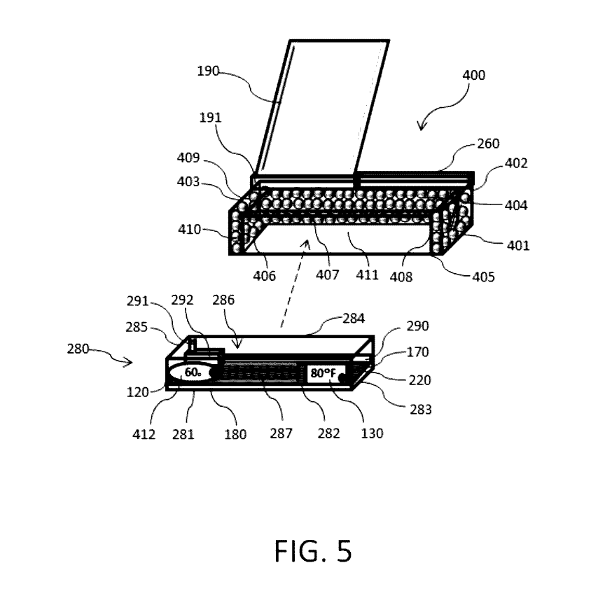

FIG. 5 is a perspective view of one insulated modular drawer embodiment 400 of the present invention and the accompanying modular drawer 280 in an unassembled state.

As shown in FIG. 5, the insulated modular drawer embodiment 400 comprises an external first wall 401, an external second wall 402, an external third wall 403, an overhead wall 404, and a support base 405. The insulated modular drawer embodiment 400 further comprises an internal first wall 406, an internal second wall 407, and an internal third wall 408. An insulation cavity 409 exists between the external and internal walls. The enclosed insulation cavity 409 holds multiple insulation beads 410. The mirror 190 is held in upright position by the mirror holder 191 which is attached to the overhead wall 404. The electronic device holder 260 is also attached to the overhead wall 404.

As shown in FIG. 5, the modular drawer 280 comprises a support base 281, a first wall 282, a second wall 283, a third wall 284, and a fourth wall 285. The first wall 282, second wall 283, third wall 284, and fourth wall 285 extend upward from a first side of the support base 281. The internal storage compartment 286 is the area that holds cosmetics products. Modular drawer 280 has a drawer handle 287 on wall first wall 282 used to pull out the modular drawer 280 from the insulated external modular drawer encasement 400. The modular drawer 280 contains a perforated desiccant holder 170 that sits on a circumferential ledge 220 above the support base 281 creating an airflow space 180 between the desiccant holder 170 and the support base 281.

FIG. 5 shows an electronically controlled timer 120 and thermometer 130 affixed to the first wall 282 of the modular drawer 280.

FIG. 5 shows a dotted arrow to indicate the direction in which the modular drawer 280 assembles into the drawer compartment 411 to create one insulated modular drawer embodiment 400 of the present invention.

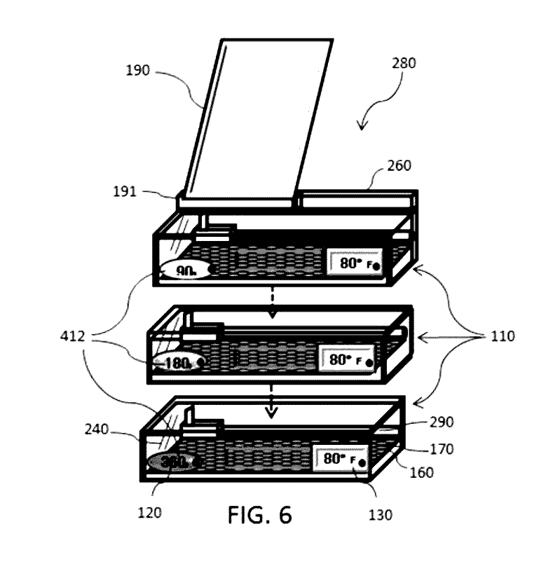

FIG. 6 is a perspective view of one way that modular drawers 280 can be stacked to create a plurality of multiple storage compartments 110. FIG. 6 shows a modular drawer 280 configuration which includes a mirror 190, mirror holder 191, and electronic device holder 260. The modular drawers 280 further comprise of electronically controlled timers 120, thermometers/thermal sensors 130, and ultraviolet germicidal irradiation lamps 290. Each modular drawer contains a desiccant sheet 171 held in place by a desiccant holder 170.

The features of the invention illustrated and described herein are the preferred embodiments. Therefore, it is understood that the appended claims are intended to cover unforeseeable embodiments with insubstantial differences that are within the spirit of the claims.

What is claimed is:

1. An apparatus for monitoring cosmetic spoilage comprising:
an organizer including a plurality of multiple storage holding compartments;
an electronically controlled timer adapted for attachment to the individual storage holding compartments;
a control on the electronically controlled timer to initiate a time period of use for the individual cosmetic storage holding compartment, and, a visual or audio indicator triggered by the electronically controlled timer notifying a user when the end use or recommended time frame of a cosmetic product stored in one the compartment has expired.

2. The apparatus according to claim 1 wherein the organizer includes a thermal sensor that measures ambient temperature and, the visual or audio indicator is responsive to temperature fluctuations.

3. The apparatus according to claim 1 made out of translucent materials having ultraviolet resistant tinting.

4. The apparatus according to claim 1 wherein the organizer is modular.

5. The apparatus according to claim 1 wherein the plurality of multiple storage holding compartments contain visual identifications for cosmetic categories.

6. The apparatus according to claim 1 further comprising a mirror to aid in the application of cosmetic products.

7. The apparatus according to claim 1 which includes an electronic device holder.

8. The apparatus according to claim 1 comprising:
a bottom portion of the organizer below the floor of the plurality of cosmetic compartments,
a gap between the bottom portion of the organizer and the floor of the plurality of cosmetic compartments configured to hold a desiccant article in the gap between the bottom portion of the organizer and the floor of the plurality of cosmetic compartments, and, the floor of the plurality of cosmetic compartments is perforated to permit air flow between the perforated floor of the bottom of the plurality of cosmetic compartments and the bottom portion of the organizer supporting upon it the desiccant article.

9. The apparatus according to claim 1 further comprising a desiccant in each cosmetic product compartment.

10. The apparatus according to claim 1 further comprising ultraviolet germicidal irradiation lighting.

11. The apparatus according to claim 1 which is insulated.

12. The apparatus according to claim 12 which contains coolant beads.
